(19) United States
(12) Patent Application Publication
(10) Pub. No.: US 2019/0341138 A1
(43) Pub. Date: Nov. 7, 2019

(54) DEOPTIMIZED SHOPPING ROUTE FOR ACHIEVING USER FITNESS GOALS

(71) Applicant: Walmart Apollo, LLC, Bentonville, AR (US)

(72) Inventors: Bruce Green, Centerton, AR (US); Yurgis Mauro Bacallao, Centerton, AR (US); Omar Cerda, Centerton, AR (US); Arnabh Bhaumik, Bentonville, AR (US)

(21) Appl. No.: 16/402,392

(22) Filed: May 3, 2019

Related U.S. Application Data
(60) Provisional application No. 62/667,746, filed on May 7, 2018.

(51) Int. Cl. G16H 20/30; G06Q 30/06; G16H 20/60; H04W 4/021; H04W 4/024

(52) U.S. Cl. CPC G16H 20/30; G06Q 30/0639; H04W 4/024; G16H 20/60; H04W 4/021; G06Q 30/0631

(57) ABSTRACT

A method, a computer system and computer program product, including: establishing a communication link between at least one of the user mobile device and a user wearable computing device, and the computing system, receiving from at least one of the user mobile device and the wearable computing device: a shopping list of the user and a fitness goal of the user that the user has yet to achieve, and calculating a route throughout the shopping center that achieves or exceeds the fitness goal of the user while also guiding the user past the goods on the shopping list of goods that the user needs to purchase, wherein the route prioritizes achieving or exceeding the fitness goal of the user over an optimal route through the shopping center past the list of goods the user needs to purchase.

### RELATED APPLICATION

This invention claims priority to U.S. provisional patent application Ser. No. 62/667,746, filed May 7, 2018 and entitled “Deoptimized Shopping Route for Achieving User Fitness Goals,” the entirety of which is incorporated herein by reference.

### TECHNICAL FIELD

The present invention relates to systems and methods calculating shopping routes within a shopping center, and more specifically the embodiments of a route determination system for achieving user fitness goals while shopping.

### BACKGROUND

Health, nutrition, and fitness applications in combination with a user wearable device can be useful tools for a user achieve and maintain a healthy and active lifestyle. Reaching timed fitness goals, such as a daily steps goal, can be challenging due to busy schedules, office hours, etc. Moreover, locating items in a shopping center, especially healthy options part of a tailored meal plan, can be difficult if the user is not wholly familiar with the layout of the shopping center. Even if the user is familiar with the layout of the shopping center, items are often moved around to different locations in the shopping center.

### SUMMARY

An embodiment of the present invention related to a method, an associated computer system and computer program product, for generating a health and fitness integrated shopping route throughout a shopping center, the method comprising: establishing, by a processor of a computing system, a communication link between at least one of the user mobile device and a user wearable computing device, and the computing system, over a network, wherein the user wearable computing device has at least one sensor for tracking a movement of the user; in response to establishing the communication link: receiving, by the processor, from at least one of the user mobile device and the wearable computing device: a shopping list of the user and a fitness goal of the user that the user has yet to achieve, and calculating a route throughout the shopping center that achieves or exceeds the fitness goal of the user while also guiding the user past the goods on the shopping list of goods that the user needs to purchase, wherein the route prioritizes achieving or exceeding the fitness goal of the user over an optimal route through the shopping center past the list of goods the user needs to purchase.

An embodiment of the present invention relates to a method, and associated computer system and computer program product, for generating a health and fitness integrated shopping route throughout a shopping center, the method comprising: establishing, by a processor of a computing system, a communication link between at least one of the user mobile device and a user wearable computing device, and the computing system, over a network, wherein the user wearable computing device has at least one sensor for tracking a movement of the user, in response to establishing the communication link: receiving, by the processor, from at least one of the user mobile device and the wearable computing device: (i) a meal plan tailored to the user, (ii) a shopping list of the user synchronized with the meal plan to determine a list of goods that the user needs to purchase within the shopping center in accordance with the meal plan, and (iii) a fitness goal of the user that the user has yet to achieve, calculating, by the processor, a route throughout the shopping center that achieves or exceeds the fitness goal of the user while also guiding the user past the goods on the shopping list of goods that the user needs to purchase, wherein the route prioritizes achieving or exceeding the fitness goal of the user over an optimal route through the shopping center past the list of goods the user needs to purchase, tracking, by the processor, goods scanned using a mobile payment application for purchase by the user, determining, by the processor, that a scanned good is not on the shopping list or an approved good of the meal plan, and calculating a net caloric content between the scanned good not on the shopping list and an approved meal plan good being replaced by the scanned good, and recalculating, by the processor, the route throughout the shopping center to account for the net caloric content.

### BRIEF DESCRIPTION OF THE DRAWINGS

FIG. 1 depicts a block diagram of a route determination system, in accordance with embodiments of the present invention.

FIG. 2 depicts a schematic view of a user mobile device, having a plurality of relevant applications for the route determination system of FIG. 1, in accordance with embodiments of the present invention.

FIG. 3 depicts an optimal route for locating the needed goods in a shortest amount of time in a shortest amount of walking distance.

FIG. 4 depicts a calculated shopping route that allows a user to achieve a fitness goal by following the route, while also being led past needed shopping goods on a shopping list, in accordance with embodiments of the present invention.

FIG. 5 depicts a perspective view of a shopping cart having a computing system, in accordance with embodiments of the present invention.

FIG. 6 depicts a first updated calculated route that takes into consideration a product scanned that was not part of the user's tailored meal plan, in accordance with embodiments of the present invention.

FIG. 7 depicts a second updated calculated route that takes into consideration an additional product scanned that was not part of the user's tailored meal plan, in accordance with embodiments of the present invention.

FIG. 8 depicts a cross-section view of a wheel and a caster of the shopping cart in a first position, wherein a restrictive element in accordance with embodiments of the present invention.

FIG. 9 depicts a cross-section view of a wheel and a caster of the shopping cart, wherein the restrictive element is in a second position, in accordance with embodiments of the present invention.

FIG. 10 depicts a flow chart of a first embodiment of a method for generating a health and fitness integrated shopping route throughout a shopping center, in accordance with embodiments of the present invention.

FIG. 11 depicts a flow chart of a second embodiment of a method for generating a health and fitness integrated shopping route throughout a shopping center, in accordance with embodiments of the present invention.

FIG. 12 depicts a block diagram of a computer system for the route determination system of FIGS. 1-9, capable of implementing methods for generating a health and fitness integrated shopping route throughout a shopping center of FIGS. 10-11, in accordance with embodiments of the present invention.

### DETAILED DESCRIPTION

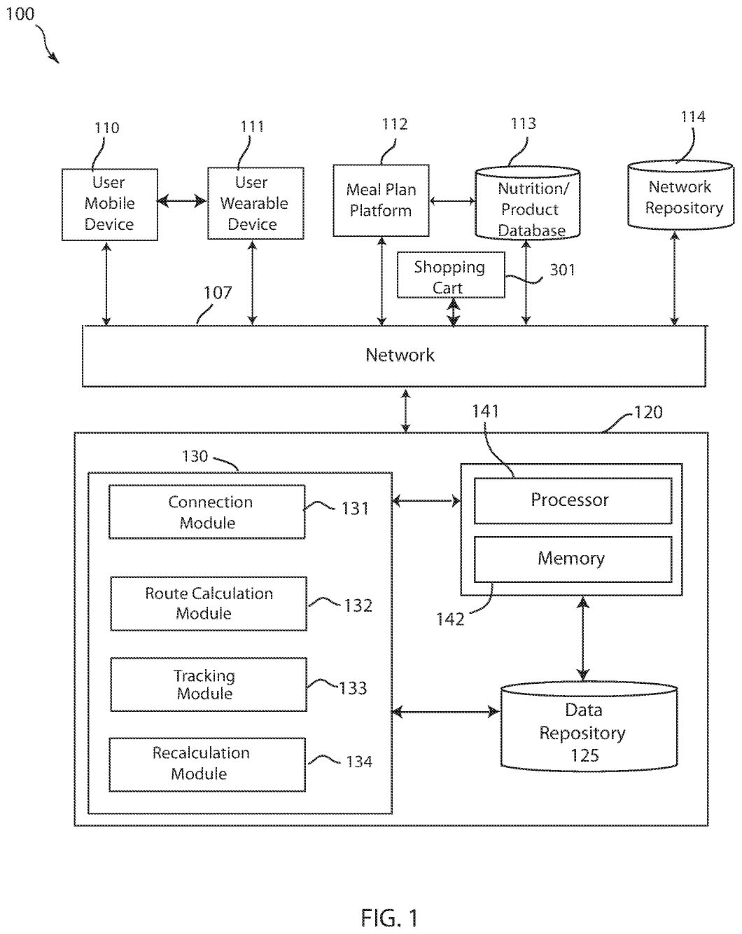

Referring to the drawings, FIG. 1 depicts a block diagram of a route determination system 100, in accordance with embodiments of the present invention. Embodiments of the route determination system 100 may be a system for calculating routes through a shopping center or similar retail environment, wherein the route is de-optimized with respect to locating certain goods needed for purchase, and optimized to achieve, exceed, or progress toward a user fitness goal. As explained in greater detail below, the route may be recalculated based on actions taken by the user during shopping. Embodiments of the route determination system 100 may be useful for users seeking to achieve, exceed, or progress toward one or more fitness goals, while being guided along aisles of a shopping environment to exact locations of needed goods. For example, the route determination system 100 may allow users to both reach fitness goals and locate needed goods with a purpose. Further, the route determination system 100 may track goods purchased by user with a mobile payment application and adjust the route accordingly. Embodiments of a shopping center may be a retailer, a salesfloor, a retail environment, a grocery store, a supercenter, a home goods store, a home improvement store, a festival, a mall, a fair, a market, and like, or any environment where it may be helpful to know a location of goods, items, objects, etc. within the environment. Embodiments of goods needed for purchase may be goods, objects, groceries, and the like, which may be offered for sale or otherwise displayed within the shopping center; the goods needed for purchase may be added to a shopping list of a user (e.g. via a retailer application or dedicated shopping list application). Embodiments of a user fitness goal may be a daily or otherwise fitness-related goal, such as daily steps, calories burned, minutes exercised, time spent at given heartrate, carbohydrates burned, fat burned, etc.

Embodiments of the route determination system 100 may be a de-optimized route guidance system, a fitness goal achievement system a shopping assistant, a fitness and shopping assistant, a product tracking and fitness system, a user nutrition and fitness system, a meal plan adviser and good locating system, combinations thereof, and the like. Embodiments of the route determination system 100 may include a computing system 120. Embodiments of the computing system 120 may be a computer system, a computer, a server, one or more servers, a backend computing system, and the like, which connect to user mobile device 110, user wearable device 111, and shopping cart 301. In other embodiments, the computing system 120 may be a computing system mounted to, attached to, coupled to, or otherwise a part of a shopping cart or similar shopping instrument used to push through the shopping center and temporarily store goods selected by the user. For instance, embodiments of the computing system 120 may be an onboard computing system of a “smartcart” used by users during a shopping experience.

Furthermore, embodiments of route determination system 100 may include a user device 110, a user wearable device 111, a shopping cart 301 a meal plan platform 112, and a nutrition/product database 113 that are communicatively coupled to the computing system 120 over a computer network 107. For instance, information/data may be transmitted to and/or received from the user device 110, the user wearable device 111, the shopping cart 301, the meal plan platform 112, and the nutrition/product database 113 over a network 107. A computer network 107 may be the cloud. Further embodiments of computer network 107 may refer to a group of two or more computer systems linked together. Computer network 107 may be any type of computer network known by individuals skilled in the art. Examples of computer network 107 may include a LAN, WAN, campus area networks (CAN), home area networks (HAN), metropolitan area networks (MAN), an enterprise network, cloud computing network (either physical or virtual) e.g. the Internet, a cellular communication network such as GSM or CDMA network or a mobile communications data network. The architecture of the computer network 107 may be a peer-to-peer network in some embodiments, wherein in other embodiments, the computer network 107 may be organized as a client/server architecture.

In some embodiments, the computer network 107 may further comprise, in addition to the computing system 120, a connection to one or more network-accessible knowledge bases 114, which are network repositories containing information of the user, product information, meal plan information, fitness information, location information, user activity, user preferences, network repositories or other systems connected to the computer network 107 that may be considered nodes of the computer network 107. In some embodiments, where the computing system 120 or network repositories allocate resources to be used by the other nodes of the computer network 107, the computing system 120 and network-accessible knowledge bases 114 may be referred to as servers.

The network-accessible knowledge bases 114 may be a data collection area on the computer network 107 which may back up and save all the data transmitted back and forth between the nodes of the computer network 107. For example, the network repository may be a data center saving and cataloging user fitness and shopping activity data, user meal plan data, user nutrition and diet data, location data, user preference data, and the like, to generate both historical and predictive reports regarding a particular user's eating, shopping, and fitness behavior, and the like. The reports may be used to generate rewards, such as coupons sent to the users, or virtual reward tokens accumulated in a user account, or an electronic coupon emailed other otherwise transferred to the user. In some embodiments, a data collection center housing the network-accessible knowledge bases 114 may include an analytic module capable of analyzing each piece of data being stored by the network-accessible knowledge bases 114. Further, the computing system 120 may be integrated with or as a part of the data collection center housing the network-accessible knowledge bases 114. In some alternative embodiments, the network-accessible knowledge bases 114 may be a local repository that is connected to the computing system 120.

Embodiments of the user mobile device 110 may be a computing device, a mobile computer, a cell phone, a mobile computing device, a tablet computer, a virtual assistant device, a smart speaker, a smartphone, and the like. The user mobile device 110 may include hardware functionality such as a speaker for emitting a sound, a vibration motor for creating vibrations, a display for displaying images, videos, pictorial sequences, etc., a light emitting element for emitting a light, a receiver for receiving communications, a transmitter for transmitting signals, and other similar features and hardware of a smartphone, smartwatch, cell phone, tablet computer, and the like. Furthermore, embodiments of the user wearable device 111 may be a wearable computing device, a wearable sensor, a wearable electronic device, a smartwatch, an internet connected apparel, shoes having one or more sensors for transmitting data, smartglasses, a heart rate monitor, a fitness tracking device, a steps tracker, and the like, or any device having at least one sensor for capturing a fitness-related data point that can transmit or otherwise send the fitness-related data to the user mobile device 110 and/or the computing system 120. The user wearable computing device 111 may include hardware functionality such as a speaker for emitting a sound, a vibration motor for creating vibrations or other haptic feedback, a display for displaying images, videos, etc., a light emitting element for emitting a light, a receiver for receiving communications, a transmitter for transmitting signals over network 107 or other short range communication network, a sensor for capturing fitness-related data (e.g. a movement of the user, a calories burned by the user, and the like), a gyroscope, an accelerometer, a heart rate monitor, and other similar features and hardware of a user wearable computing device.

Referring still to FIG. 1, embodiments of the route determination system 100 may include a meal plan platform 112. Embodiments of the meal plan platform 112 may be communicatively coupled to the computing system 120 over computer network 107, and may be communicatively coupled to the user mobile device 110 and/or the user wearable device 111. Embodiments of the meal plan platform 112 of the route determination system 100 depicted in FIG. 1 may be one or more meal planning services, engines, platforms, system, websites, applications, etc. Moreover, embodiments of meal plan platform 112 may be one or more websites, applications, databases, storage devices, repositories, servers, computers, engines, and the like, that may plan, organize, schedule, suggest, create, etc. individual meals for a user. The meal planning platform 112 may plan meals for each day, each week, month, etc. for the user to follow. The meal planning platform 112 may provide detailed recipes, meal ingredients, portions, cooking instructions, shopping lists for preparing/making each meal, and the like. Embodiments of the meal plan platform 112 may tailor a meal plan for the user, depending on the user's preferences, weight loss goal, health goals, lifestyle, and the like. The meal planning platform 112 may be used by the user to manage weight and other personal reasons, wherein the meal planning platform 112 may provide detailed shopping lists for the user to purchase goods at a shopping center to accomplish and follow through with the scheduled meal plan created by the meal planning platform 112. Embodiments of the meal plan platform 112 may be a subscription based service, offering a mobile application downloadable to the user mobile device 110 and/or the user wearable device 111, wherein the meal planning platform 112 may be integrated with other software applications of the user mobile device 110 and/or the user wearable device 111, such as a calendar application, a shopping list application, a fitness/activity application, a retailer application, a mobile payment application, and the like. The meal planning platform 112 may be accessed over network 107 by the computing system 120 or may be accessed by the computing system via the user mobile device 110 and/or the user wearable device 111, and may be managed and/or controlled by a third party.

Embodiments of shopping cart 301 may be a conventional shopping cart configured to store, hold, receive, transport, etc. objects such as store goods when the user is shopping. Embodiments of the shopping cart 301 may additionally have an onboard computer for receiving instructions from the computing system 120 and transmitting data to and from the computing system 120. The onboard computer may be ruggedized, and maintained within a housing or other protecting element to shield the onboard computer from damage. The onboard computer may control or otherwise operate: a display for displaying a calculated route, a user interface for inputting commends (e.g. touch screen) a speaker for emitting sound, an actuator for adding or reducing a resistance applied to at least one wheel of the shopping cart 301, a network interface controller, and the like. Embodiments of the onboard computer of shopping cart 301 may not be running a shopping and fitness assistance application 130 of the route determination system 100, which is a software application run by the computing system 120.

Furthermore, embodiments of the computing system 120 may be equipped with a memory device 142 which may store various data/information/code, and a processor 141 for implementing the tasks associated with the route determination system 100. In some embodiments, a shopping and fitness assistance application 130 may be loaded in the memory device 142 of the computing system 120. Embodiments of the shopping and fitness assistance application 130 may be an interface, an application, a program, a module, or a combination of modules. In an exemplary embodiment, the shopping and fitness assistance application 130 may be a software application running on one or more back end servers, servicing a user device 110, a user wearable device 111, or a computer associated with a shopping cart (e.g. “smartcart”), wherein a user interface portion of the software application may also run on the user device 110, user wearable device 111, and/or an internet-connected shopping cart.

The shopping and fitness assistance application 130 of the computing system 120 may include a connection module 131, a route calculation module 132, a tracking module 133, and a recalculation module 134. A “module” may refer to a hardware-based module, software-based module or a module may be a combination of hardware and software. Embodiments of hardware-based modules may include self-contained components such as chipsets, specialized circuitry and one or more memory devices, while a software-based module may be part of a program code or linked to the program code containing specific programmed instructions, which may be loaded in the memory device of the computing system 120. A module (whether hardware, software, or a combination thereof) may be designed to implement or execute one or more particular functions or routines.

Embodiments of the connection module 131 may include one or more components of hardware and/or software program code for establishing a communication link between at least one of the user mobile device 110 and a user wearable computing device 111, and the computing system 120, over a network 107. For instance, the connection module 131 may be configured to connect to the user mobile device 110 and/or a user wearable computing device 111, such that data can be wirelessly transmitted between the user mobile device 110 and/or a user wearable computing device 111 and the computing system 120. The user mobile device 110 and/or a user wearable computing device 111 may connect to the computing system 120 over the Internet, or may be linked or paired to the computing system 120. The user mobile device 110 and/or a user wearable computing device 111 may be paired to the computing system using near field communication (NFC) or may be paired to the computing system 120 over a short-range communication network, such as Bluetooth®. The connection module 131 may manage the connections and communication links between the user mobile device 110 and/or a user wearable computing device 111 and the computing system 120. In an exemplary embodiment, the computing system 120 may be physically located on a shopping cart within the shopping center, as described in greater detail infra, in which the user mobile device 110 and/or a user wearable computing device 111 may be paired with the computing system 120 of the shopping cart. In another exemplary embodiment, the computing system 120 may be one or more remote servers, wherein the user mobile device 110 and/or a user wearable computing device 111 wirelessly connect to the computing system via a wireless network such as a cellular network or a wi-fi network within the shopping center.

In in response to establishing the communication link (e.g. connecting via Internet or pairing), the computing system 120 may receive from at least one of the user mobile device 110 and the user wearable computing device 111 user-specific information/data. User-specific information/data may be a meal plan tailored to the user, a shopping list of the user, and a fitness goal information of the user. For instance, in response to detecting that user mobile device 110 and/or a user wearable computing device 111 are wirelessly connected to the computing system 120, the connection module 131 of the computing system 120 may request the user-specific information from the user mobile device 110 and/or a user wearable computing device 111. The user-specific information may be transmitted to the computing system 120 in response to the request from the computing system 120, either from the user mobile device 110 or the user wearable computing device 111, or both of the user mobile device 110 and the wearable computing device 111. The user mobile device 110 and the user wearable computing device 111 may be paired together over a short-range communication network, and may share information/data between each other to effectively respond to the request for the user-specific data/information, and communicate therebetween.

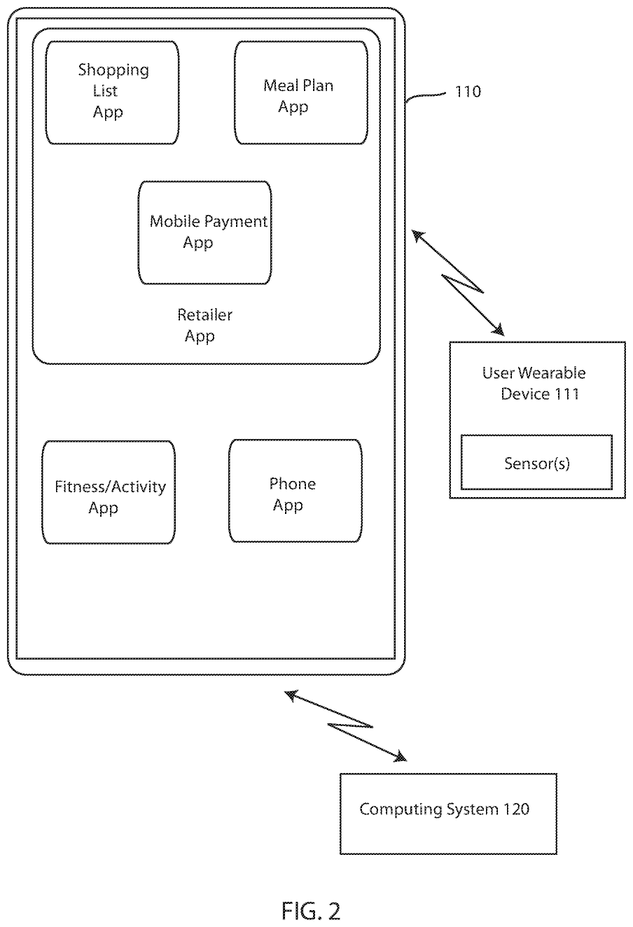

The user-specific information may be received from the user mobile device 110, which has various, relevant application loaded thereon. FIG. 2 depicts a schematic view of a user mobile device 110, having a plurality of relevant applications for the route determination system 100 of FIG. 1, in accordance with embodiments of the present invention. The user mobile device 110 may be wirelessly connected to the computing system 120 and the user wearable computing device 111, as shown in FIG. 2. Embodiments of the user mobile device 110 may include a retailer software application, which may have various features included therein, such as a shopping list application, a meal plan application, a fitness/activity application, and a mobile payment application. For example, a software application downloadable to user mobile devices 110 offered by a retailer may include a shopping list feature implemented by the shopping list application, a meal planning service/feature implemented by the meal plan application, a fitness/activity feature implemented by the fitness/activity application, and mobile payment feature (e.g. application allowing a mobile payment of an item scanned by a camera). The single retailer application may offer these features, wherein one or more backend servers service the various functions of the shopping list application, the meal plan application, the fitness/activity application, and the mobile payment application. In alternative exemplary embodiment, the retailer application may include one or more of the shopping list application, the meal plan application, the fitness/activity application, and the mobile payment application. FIG. 2 depicts a retailer application providing the shopping list application, the meal plan application, and a mobile payment application, wherein the fitness/activity application is a separate application downloadable to the user mobile device 110, serviced by other backend servers. Various combinations of applications within the retailer application may be suitable for implementing the route determination system 100, including a retailer application that only supports the mobile payment application, while other third-party application developers operate and maintain the shopping list application, the meal plan application, and the fitness/activity application.

Referring still to FIGS. 1 and 2, embodiments of the connection module 131 may request, obtain, or otherwise receive the user-specific information from the user mobile device 110, the user wearable device 111 and/or the meal plan platform 113, running the shopping list application, the meal plan application (e.g. which may be associated with the meal plan platform 112), the fitness/activity application, and the mobile payment application. The computing system 120 may receive a meal plan tailored to the user. The meal plan tailored to the user may be received from the user mobile device 110 or the user wearable device 111 directly, via interfacing with the meal plan application running on the devices 110, 111, or may be received from the meal plan platform 112 in response to the connection module 131 interfacing with or otherwise accessing the meal plan platform 112 with required user permissions and/or login information. The meal plan tailored to the user may be a daily, weekly, monthly schedule of meals, including a detailed breakdown of what the user is scheduled to eat for breakfast, lunch, dinner, snacks, desserts, etc., at various points in the day. The meal plan tailored to the user may also include nutrition information regarding each ingredient used in the individual meals, such as calories, grams of fat, sugar, carbohydrates, and the like. The computing system 120 may also receive a shopping list of the user including goods that the user needs to purchase. The shopping goods that the user desires to purchase may be goods (e.g. food, ingredients, etc.) that the user has preselected to be placed on a list for purchasing using a shopping list application or retail application offering a shopping list creation feature. In an exemplary embodiment, the list of shopping goods of the user may be synchronized with the meal plan information to automatically determine a list of goods that the user needs to purchase within the shopping center in accordance with the meal plan. For instance, the meal plan platform 112 servicing the user mobile device 110 may automatically generate a shopping list with items the user needs to purchase for cooking a meal that day, next day, the next couple of days, or a week or more, in accordance with the tailored meal plan. The automatically generated shopping list may be sent to the computing system 120 in response to the request from the connection module 131. The computing system 120 may also receive user fitness information from the user mobile device 110 and/or the user wearable computing device 111. For example, the connection module 131 may request, obtain, or otherwise receive fitness and activity information of the user, which may include one or more fitness goals of the user that the user has yet to achieve. The fitness or activity goal may be a daily step total, a total number of minutes exercising, a number of calories burned, and the like. The connection module 131 may analyze the fitness and activity information/data to determine whether a particular goal has not been achieved at a time of establishing the communication link with the computing system 120.

Although the connection module 131 may request the user-specific information in response to detecting that the devices 110, 111 have established a communication like, the devices 110, 111 may automatically send the user-specific information to the computing system 120 upon the communication link(s) being established.

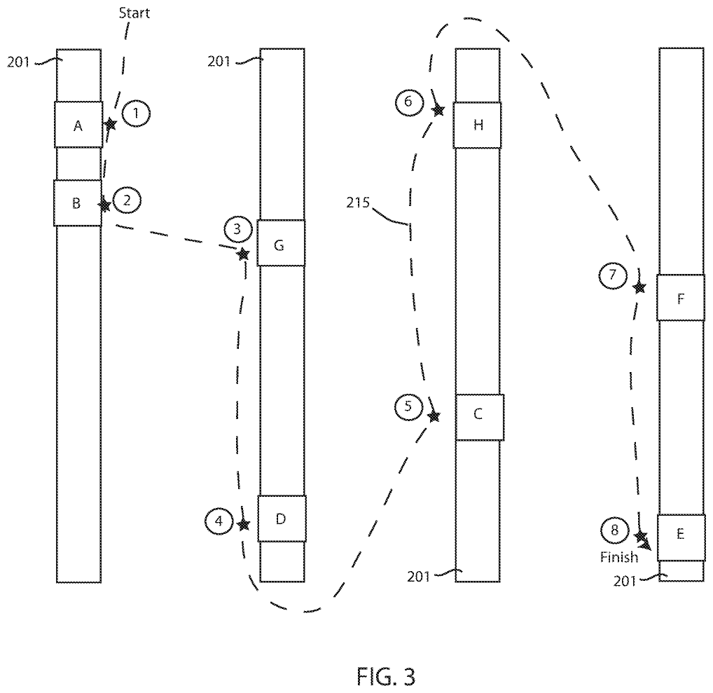

Referring back to FIG. 1 embodiments of the computing system 120 may include a route calculation module 132. Embodiments of the route calculation module 132 may include one or more components of hardware and/or software program code for calculating a route throughout the shopping center that achieves, exceeds, or progresses toward achieving the fitness goal of the user while also guiding the user past the goods on the shopping list of goods that the user needs to purchase. The route calculation module 132 may calculate a route that prioritizes achieving, exceeding, or progressing towards achieving the fitness goal of the user over an optimal route through the shopping center past the list of goods the user needs to purchase. For instance, embodiments of the route calculation module 132 may determine a path through the shopping center that may be de-optimal or de-optimized for locating the needed goods in a shortest amount of time in a shortest amount of walking distance. FIG. 3 depicts an optimal route for locating the needed goods in a shortest amount of time in a shortest amount of walking distance. The optimal shopping route 215 for finding needed shopping goods A-H in the shortest amount of time and the least amount of walking may be optimal for shopping efficiency and time savings. In FIG. 3, the optimal shopping route 215 to pick up and purchase goods A-H may be to make a first stop at good A from a starting point (labeled “start”), then a direct path to a second stop at good B in a same aisle, then a direct path to a third stop at good G in the same aisle, then a direct path to a fourth stop at good D in the same aisle, then a direct path to a fifth stop at good C in the next, adjacent aisle, then a direct path to a sixth stop at good H in the same aisle, then a direct path to a seventh stop at good F in the next, adjacent aisle, and then finally a direct path to an eighth stop at good E in the same aisle to finish the shopping route (labeled “finish”). However, the shopping and fitness assistance application 130 of computing system 100 of the route determination system 100 prioritizes achieving the fitness goals of the user over a speed or efficiency of a route through the shopping center that optimally takes the user past the shopping goods on a user shopping list. Rather, the route calculation module 132 may determine the optimal route for achieving a fitness goal of the user, which may be purposefully de-optimal for walking through the shopping center to locate and pick up goods in a most time-saving manner.

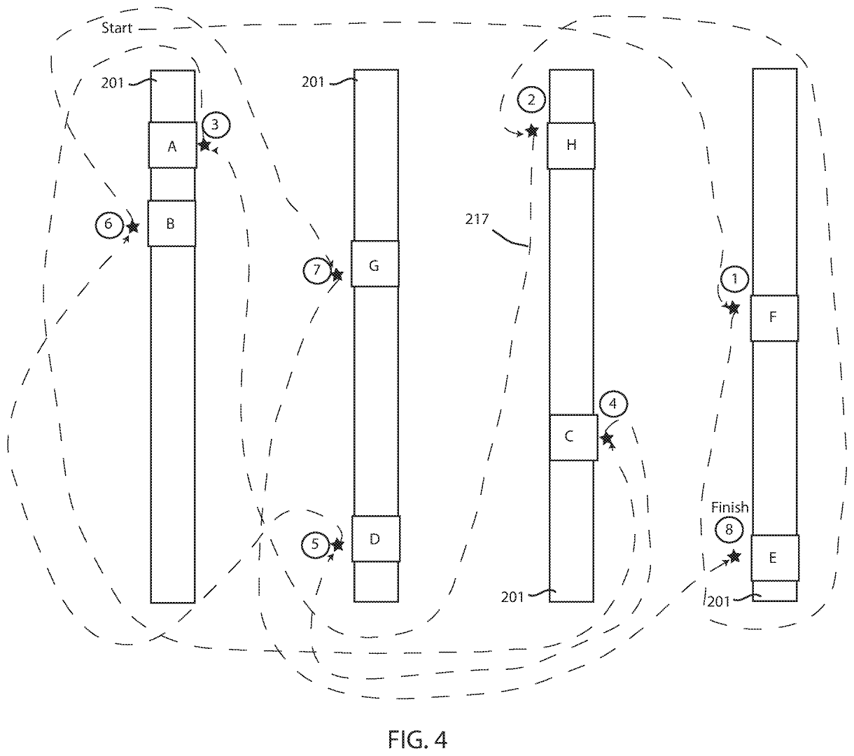

FIG. 4 depicts a calculated shopping route 217 that allows a user to achieve a fitness goal by following the route, while also being led past needed shopping goods on a shopping list, in accordance with embodiments of the present invention. The calculated shopping route 217 calculated by the route calculation module 132 may be longer in total distance traveled (e.g. walked) than the optimal shopping route 215, so the user can achieve a fitness goal, such as a total number of daily steps, calories burned, etc. Additionally, the calculated shopping route 217 may intentionally guide the user past goods A-H needed on the shopping list to guide the user along a longer path to a particular good. In FIG. 4, the calculated shopping route 217 to pick up and purchase goods A-H may be to make a first stop at good F several aisles from a starting point (labeled “start”), then to a second stop at good H in in the next, adjacent aisle but taking a longer, circuitous route around a back side of the aisle containing good F. From there, the calculated shopping route 217 guides the user to a third stop at good A which is two aisles from the previous good, H, and then to a fourth stop at good C which is two aisles from the previous good, H, and back in the direction the user just came from. Next, the calculated shopping route 217 guides the user to a fifth stop at good D in the next, adjacent aisle, then a direct path to a sixth stop at good B in the next aisle, then a direct path to a seventh stop at good G in the next, adjacent aisle, and then finally a direct path to an eighth stop at good E several aisles over to finish the shopping route (labeled “finish”).

A difference between a total distance of the calculated shopping route 217 of FIG. 4 and a total distance of the optimal shopping route 215 in FIG. 3 may be equivalent to or may correspond to a number of steps required by the user to achieve the user fitness goal. In other words, the route calculation module 132 may determine a distance (e.g. inches, feet, yards, meters, etc.) that may be equivalent to a number of steps the user has to take to achieve the user's fitness goals, and add the distance to the optimal shopping route 215. In an exemplary embodiment, the added distance may be divided evenly between the number of goods on the user shopping list. For example, if the user needs to purchase 7 items at the shopping center, and needs 1000 steps to achieve a daily fitness goal, the route calculation module 132 may first calculate the optimal route 215, determine the total needed distance to add to the distance of the optimal route 215, and then divide the total distance by the number of goods, 7. In another exemplary embodiment, the added distance may be added completely randomly, regardless of the number of items on the user shopping list. For instance, the calculated route 217 may take the user past the first six of seven items, and then take the user on a very long path to the seventh and final item. Furthermore, embodiments of the route calculation module 132 may calculate the calculated route 217 without first calculating the optimal route 215. The route calculation module 132 may determine the number of steps the user needs to complete the daily fitness goal, in view of the goods that the user needs, and create one or more different routes that would accomplish the same goal. The user may be presented with several options, or the route calculation module 132 may automatically select a calculated route 217 based on a confidence score that the user will reach the fitness goal, or based on another factor, such as less stops in the beginning or more stops at the end, and the like.

Furthermore, embodiments of the route calculation module 132 may calculate the calculated route 217 based on fitness goals other than daily steps. For example, the route calculation module 132 may calculate a route that will allow the user to burn 350 calories. In other embodiments, the route calculation module 132 may calculate the calculated route 217 based on a user-selected portion of a fitness goal. For example, the connection module 131 may determine that the user has a daily goal of 10000 steps, and at the time of establishing the connection/communication link with the computing system 120, the user still needs 7,000 steps (e.g. remaining steps to achieve goal). The user may elect to have a route 217 calculated that will accomplish 2000 steps (e.g. portion of steps of the remaining steps) towards the user's fitness goal during a shopping experience, as opposed to achieving the full goal of 10000 steps and needing a route 217 to be calculated that would correspond to 7000 steps. Accordingly, the route calculation module 132 may calculate a calculated route 217 that helps the user progress toward the daily fitness goal, which may result in a longer total shopping time than without the calculated route 217 (e.g. taking an optimal shopping route 215 or close to optimal route), but may also result in less total shopping time than if the user achieved the final fitness goal. Further, embodiments of the route calculation module 132 may calculate a route that will allow the user to exceed a fitness goal. For example, the connection module 131 may determine that the user has a daily goal of 10000 steps, and at the time of establishing the connection/communication link with the computing system 120, the user still needs 7,000 steps (e.g. remaining steps to achieve goal). The user may elect to have a route 217 calculated that will exceed the 10000 steps goal by 1000 steps, as opposed to only achieving the full goal of 10000 steps (i.e. the users needs a route 217 to be calculated that would correspond to 8000 steps instead of 7000 steps). Accordingly, the route calculation module 132 may calculate a calculated route 217 that helps the user exceed daily fitness goals, which may result in a longer total shopping time than without the calculated route 217 (e.g. taking an optimal shopping route 215 or close to optimal route), but may lead to more exercise for the user, while still also purchasing the goods on the user's shopping list. The computing system may generate rewards, such as coupons, for exceeding and/or achieving the fitness goal of the user.

Embodiments of the route calculation module 132 may calculate the deoptimized shopping route 217 (or optimized combination of fitness goal and shopping route 217) using known floor plans of shopping centers, data transmitted from one or more beacons placed within the shopping center, inventory databases, inventory management software applications, GPS information, location based alerts, and the like, to provide precise locations of goods within the shopping center. For example, a map or floor plan may be stored by the computing system 120 or may be uploaded to the computing system 120. The route calculation module 132 of the computing system 120 may instruct the user mobile device 110 and/or wearable device 111 to display a map, floor plan, schematic plans, etc. of the shopping center, and then may superimpose a route, path, line, colored line, etc. onto the displayed map area of the devices 110, 111. Other methods to display maps, routes, turn-by-turn directions, etc. to the mobile devices 110, 111 may be suitable for the route determination system 100.

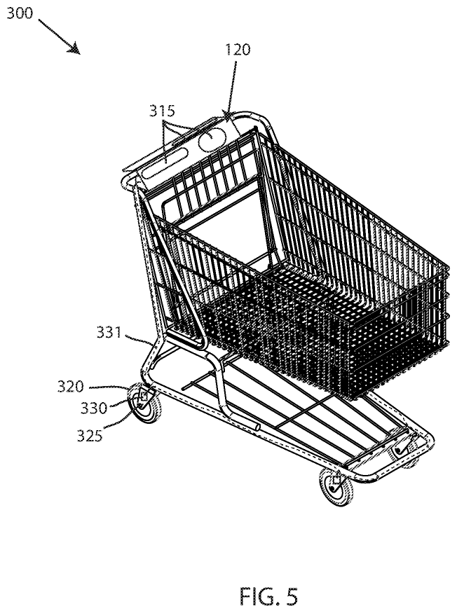

Referring now to FIG. 5, which depicts a perspective view of a shopping cart 300 having a computing system 120, in accordance with embodiments of the present invention. In some embodiments of the route determination system 100, the computing system 120 may be associated with the shopping cart 300. For example, the computing system 120 may be physically attached to the shopping cart 300. Embodiments of shopping cart 300 may share the same structural and functional aspects as shopping cart 301, but may include the computing system 120 instead of the onboard computer with limited functionality as described supra. Embodiments of the shopping cart 300 may be a smartcart, a shopping instrument, a carriage, a rolling basket, a cart, and the like, which may be configured to store, contain, hold, collect, accommodate, receive, etc. one or more objects. Embodiments of the shopping cart 300 may include the computing system 120 coupled, mounted, affixed, attached, secured, fastened, etc. to a portion of the shopping cart 300. In an exemplary embodiment, the computing system 120 may be mounted to the shopping cart 300 proximate or otherwise near a handle of the shopping cart. The computing system 120 may be enclosed or otherwise housed by a housing or other protective structure that can be physically attached to the shopping cart 300. The computing system 120 and other electronic components of the shopping cart 300 may be powered by a power source, such as one or more electric battery, one or more rechargeable battery, or one or more conventional batteries. Moreover, embodiments of the computing system 120 may include one or more user interfaces 315 for allowing users to interact with the computing system. For example, a user may select various options from the user interface 315 that corresponds to a particular fitness goal that the user would like to achieve, or which route from a plurality of possible routes the user prefers. The user interface(s) 315 may include one or more displays for displaying the route 217 and providing directions to the user for walking around shopping center. In an exemplary embodiment, a user may enter the shopping center, grab a cart, and be prompted to connect with the computing system 120. The prompt may be displayed on one of the user interface 315 displays, and the user may select (e.g. by touching the display screen) to agree to connect with the computing system 120 of the shopping cart 300. Once connected, the computing system 120 may display the calculated route 217 for the user to follow.

Referring back to FIG. 1, embodiments of the computing system 120 may include a tracking module 133. Embodiments of the tracking module 133 may include one or more components of hardware and/or software program for tracking goods scanned using a mobile payment application for purchase by the user. Embodiments of the tracking module 133 may track, monitor, analyze, etc. each scan of a product by the user using a mobile payment application loaded on the user mobile device 110 or the user wearable device 111. For example, if a user intends to purchase a good, the user may scan a bar code or other computer readable unique identifier using a camera of the user mobile device 111, and the mobile payment application will perform the necessary functions to allow the user to keep shopping and then ultimately pay for the items using the mobile payment application. The tracking module 133 may interface or otherwise communicate with the mobile payment application to obtain a data regarding the product scanned. Based on the data collected from the scan, embodiments of the tracking module 133 may determine that a scanned good is not on the shopping list or an approved good of the meal plan. For instance, embodiments of the tracking module 133 may analyze the data of a scanned good to evaluate whether the scanned good is an approved product or a part of the user's tailored meal plan. The analyzing and evaluating the data from scanning the good may be done to determine a nutritional information about the scanned goods (e.g. if the scanned good is an edible object) and/or an identifying information of the product scanned. In some embodiments, the tracking module 133 may access the nutrition/product database 113 to obtain, retrieve, or otherwise receive nutritional information about products scanned by the user. The nutrition/product database 113 may be a database storing, containing, etc. a nutritional information about products, such as a caloric content, a serving size information, an ingredients list, a composition, and the like.

Because the user's meal plan information is accessible or transmitted to the computing system 120 when the user device 110 and/or user wearable device 111 connects to the computing system 120, the tracking module 133 may compare the nutritional information scanned data with approved products which are intended to be used as ingredient for an upcoming meal of the user's meal plan. Based on the comparison, the tracking module 133 may calculate a net caloric content between the scanned good not on the shopping list and/or not an approved product for the meal plan and an approved meal plan good being replaced by the scanned good. In other words, if the user grabs 2% milk instead of the skim milk that the meal plan is calling for, the tracking module 133 may detect that the 2% milk product has been scanned, obtain the nutritional information of the 2% milk product and the nutritional information of the approved skim milk product, and calculate how many more calories consuming the 2% milk will be against consuming the skim milk, in accordance with an amount of skim milk is called for in a recipe of an upcoming meal in the user's custom meal plan. Based on the net caloric content, the route determination system 100 may adjust the calculated route to counteract the added calories by increasing a difficulty or increasing a distance of the route so that the user can burn calories equal to the net caloric content, or eat least burn a portion of the added net caloric content.

In some embodiments, the tracking module 133 may alert the user that the user has scanned an unapproved item. In response to the alert, the user may return the unapproved item and purchase the approved item. If the user ignores or dismissed the alert, the tracking module 133 may suggest, display, or otherwise provide recommendations for alternative products to the unapproved, scanned good that may result in no caloric gain or an alternative that may result in a lowest net gain caloric content, as compared to the intended item. Further, the recommendation may be displayed along with a predicted time to complete the updated route for each proposed alternative.

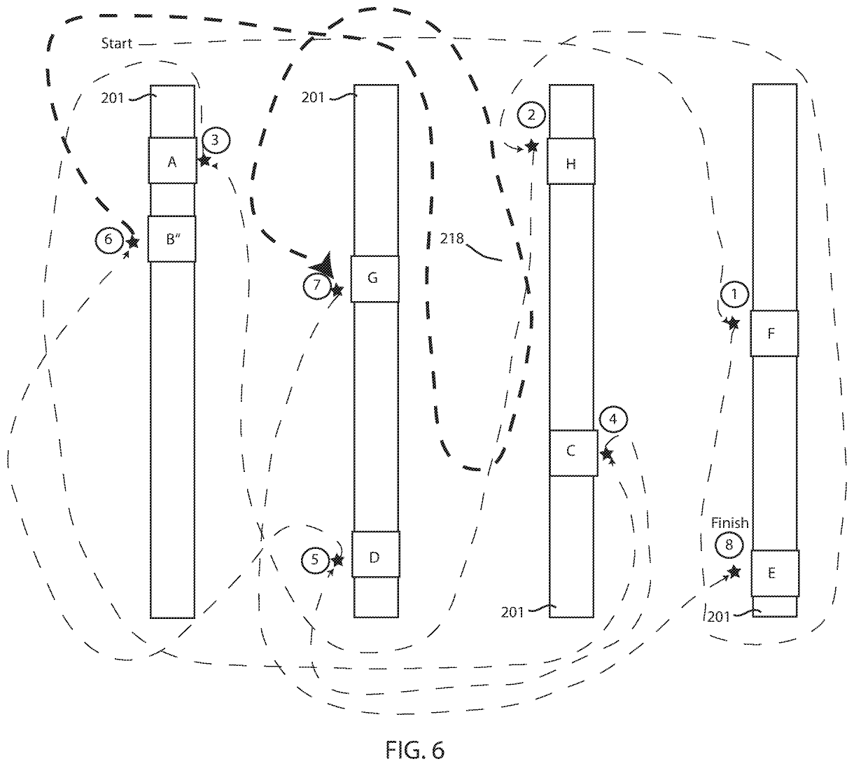

Referring still to FIG. 1, embodiments of the computing system 120 may also include a recalculation module 134. Embodiments of the recalculation module 134 may include one or more components of hardware and/or software program for recalculating the route throughout the shopping center to account for the net caloric content. For instance, embodiments of the recalculation module 134 may adjust a calculated route 217 if the user deviates from the tailored meal plan and/or the shopping list, and purchases an additional item or swaps an item for a less healthy option. The route 217 may be adjusted by adding additional distance to the total distance traveled (e.g. walked), or by increasing a difficulty by applying a restriction force against the wheel of the shopping cart 300, 301 to make it more difficult to push the cart 300, 301 along the route 217. FIG. 6 depicts a first updated calculated route 218 that takes into consideration a product scanned that was not part of the user's tailored meal plan, in accordance with embodiments of the present invention. The first updated calculated route 218 may the same as the calculated route 215 up until the sixth stop. Instead of the user grabbing good B, which is an approved ingredient of the user's meal plan (e.g. skim milk), the user grabs and scans good B″ (e.g. 2% milk). The tracking module 133 may determine that a net caloric content between the items is “X” number of calories. Embodiments of the recalculation module 134 may determine an approximate or equivalent number of steps the user would need to take to burn of “X” number of calories. Based on the number of steps required to burn off “X” number of calories, the recalculation module 134 may adjust the route 217 to the first updated calculated route 218 to account for the additional steps. In an exemplary embodiment, the recalculation module 134 may adjust the route to account for an entire number of added calories from the user deviating from the meal plan and/or shopping list. In other exemplary embodiments, the recalculation module 134 may adjust the route to account for a percentage of added calories from the user deviating from the meal plan and/or shopping list, which may be a predetermined amount or user selected via interfacing with the computing system 120. In FIG. 6, the adjusted portion of the updated route 218 is shown darker than the original calculated route 217. The adjusted route 218 adds a distance to get to the next good G on the user shopping list, which may be a direct result of the user deviating from the meal plan and/or shopping list and purchasing an additional item or deciding to purchase a less healthy option. In some embodiments, the recalculation module 134 may adjust the initially calculated route 217 to shorten the distance and/or lessen the difficulty if the user chooses to purchase/scan a healthier option, or a product that has a lower caloric content than the intended product.

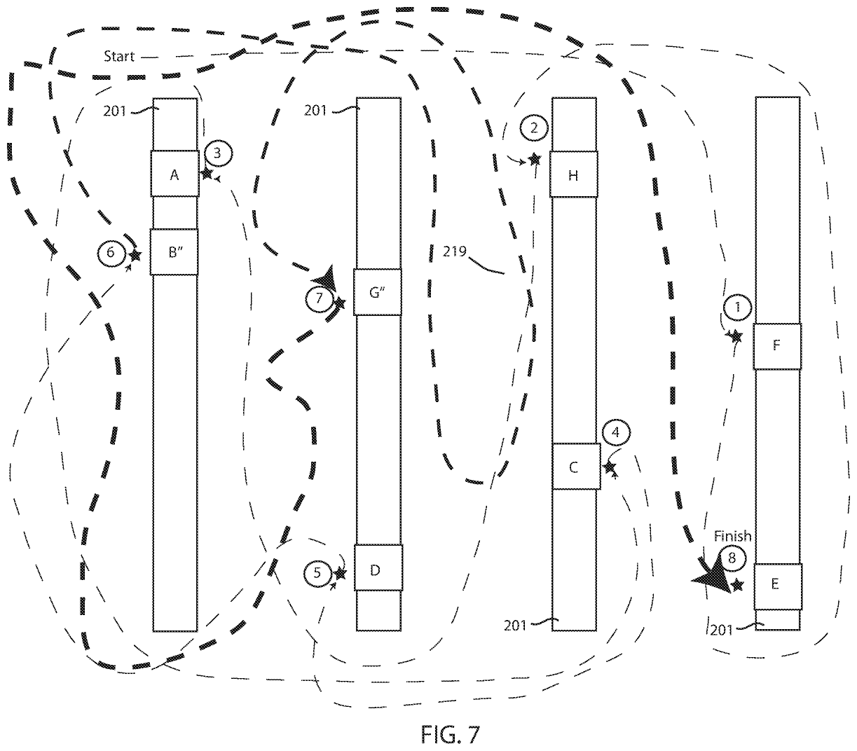

FIG. 7 depicts a second updated calculated route 219 that takes into consideration an additional product scanned that was not part of the user's tailored meal plan, in accordance with embodiments of the present invention. The second updated calculated route 219 may the same as the first updated calculated route 218 up until the seventh stop. Instead of the user grabbing good G, which is an approved ingredient of the user's meal plan (e.g. chicken breast), the user grabs and scans good G″ (e.g. chicken thigh). The tracking module 133 may determine that a net caloric content between the items is “X” number of calories. Embodiments of the recalculation module 134 may determine an approximate or equivalent number of steps the user would need to take to burn of “X” number of calories. Based on the number of steps required to burn off “X” number of calories, the recalculation module 134 may adjust the first updated calculated route 218 to the second updated calculated route 219 to account for the additional steps. In an exemplary embodiment, the recalculation module 134 may adjust the route to account for an entire number of added calories from the user once again deviating from the meal plan and/or shopping list. In other exemplary embodiments, the recalculation module 134 may adjust the route to account for a percentage of added calories from the user deviating from the meal plan and/or shopping list, which may be a predetermined amount or user selected via interfacing with the computing system 120. In FIG. 7, the adjusted portion of the updated route 218 is shown even darker than the updated portion of the first updated calculated route 218. The second adjusted route 219 adds a distance to get to the next good E on the user shopping list, which may be a direct result of the user deviating from the meal plan and/or shopping list and purchasing an additional item or deciding to purchase a less healthy option. In some embodiments, the recalculation module 134 may adjust the first updated calculated route 218 to shorten the distance and/or lessen the difficulty if the user chooses to purchase/scan a healthier option, or a product that has a lower caloric content than the intended product.

In addition to the recalculation module 134 adding total distance to the calculated routes 217, 218, 219 to account for the added number of calories based on the deviance of the user from the shopping list and/or the meal plan, the recalculation module 134 may apply a tension or restrictive force to at least one wheel of the shopping cart 300, 301. Applying a resistance to one, two, three, or even four wheels may increase a difficulty for the user to push the cart, and may make the cart 300, 301 seem heavier than the cart is to the user (e.g. the computing system 120 may simulate a cart being heavy). The increased difficulty to push the cart may require additional physical work from the user, which may cause calories to be burned by the user at a faster rate than only walking behind the cart 300, 301 and gently pushing the cart 300, 301. The increased rate of burning calories by the added restriction/tension to the wheel(s) of the cart 300, 301, may reduce a total time that the user spends completing the shopping list and achieving the user's fitness goal. The resistance can be added by any module of the computing system 120, and can be applied to: the initial calculated route 217, which may or may not affect a timing of the user reaching a fitness goal, depending on the user fitness goal that the user is trying to achieve, or to subsequent routes calculated when the user deviates from the shopping list and/or meal plan. Accordingly, the restriction may be adjusted based on an acceleration of the shopping cart, a remaining calories to be burned by the user to achieve the fitness goal, and a maximum amount of time the user wishes to spend shopping at the shopping center.

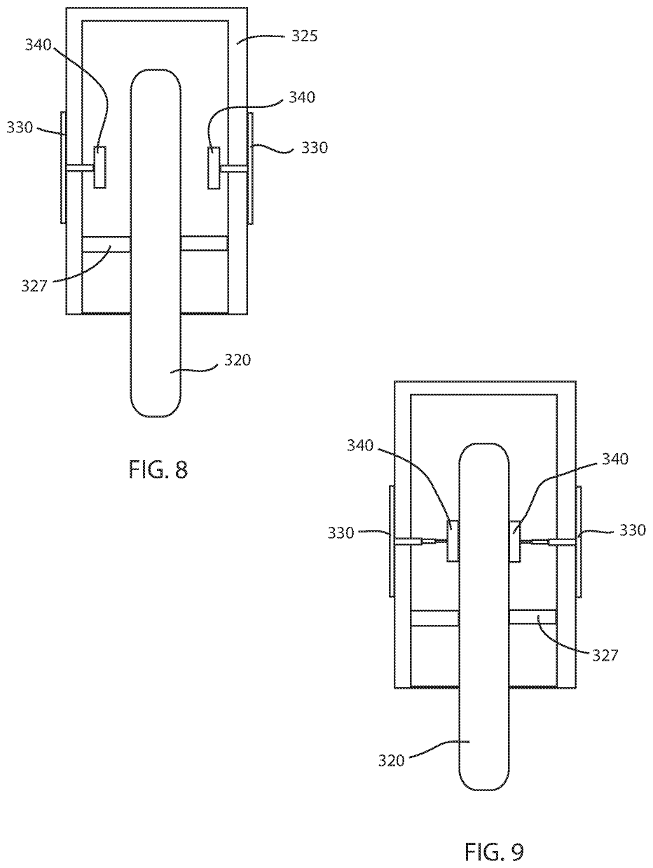

With reference back to FIG. 5, embodiments of the shopping cart 300 and shopping cart 301 may include at least one actuator 330. Embodiments of the actuator 330 may control a movement of a restriction element configured to restrict a rotation of a wheel 320 of the cart 300. The actuator 330 may be electrically connected to the computing system 120 of shopping cart 300 (as shown in FIG. 3) or may be electrically connected to the onboard computer of shopping cart 301. For instance, a cable, wire, power cord, etc., may be fed through tubing of the shopping cart 300, 301 to each actuator 330 located at each wheel 320 (shown in broken lines). In an exemplary embodiment, an actuator 330 may be placed on only a single wheel. In other embodiments, the actuator 330 may be located at more than one wheel 320. The actuator 330 may be actuated from a first position of non-engagement to a second position of engagement with the wheel 320. FIG. 8 depicts a cross-section view of a wheel 320 and a caster 325 of the shopping cart 300, 301, wherein a restrictive element 340 in a first position, in accordance with embodiments of the present invention. In the first position, the restrictive element 340 is in a position of non-engagement with a sidewall of the wheel 320, such that the wheel 320 may rotate freely about pin 327, or at least without added restriction beyond the restriction with the ground surface when moving. FIG. 9 depicts a cross-section view of a wheel 320 and a caster 325 of the shopping cart 300, 301, wherein the restrictive element 340 in a second position, in accordance with embodiments of the present invention. In the second position, the restrictive element 340 may be engaging the wheel 320, restricting, at least partially, the wheel 320 from rotating about pin 327. In an exemplary embodiment, the actuator 330 may actuate the restrictive element 340 into each position by activating a telescoping arm having the restrictive element 340 attached to and thereof. The actuator 340 may gradually increase a resistance between the wheel 320 and the restrictive element 340 by further extending the telescoping arm in a direction towards the wheel 320, and may decrease a resistance by moving the telescoping arm in a direction away from the wheel 320. The operation of the actuator 330 may be controlled by the computing system 120 mounted to the shopping cart 300, or may be controlled by an onboard computer of the shopping cart 301, which is receiving instructions from the computing system 120. Further, embodiments of the restrictive element 340 may be disc shaped, and may be comprised of a friction material, brake material, a brake pad material, and the like.

Various tasks and specific functions of the modules of the computing system 120 may be performed by additional modules, or may be combined into other module(s) to reduce the number of modules. Further, embodiments of the computer or computer system 120 may comprise specialized, non-generic hardware and circuitry (i.e., specialized discrete non-generic analog, digital, and logic-based circuitry) (independently or in combination) particularized for executing only methods of the present invention. The specialized discrete non-generic analog, digital, and logic-based circuitry may include proprietary specially designed components (e.g., a specialized integrated circuit, such as for example an Application Specific Integrated Circuit (ASIC), designed for only implementing methods of the present invention). Moreover, embodiments of the route determination system 100 offers a method for generating a health and fitness integrated shopping route throughout a shopping center using a shopping and fitness application 130. The route determination system 100 may be individualized to each user/user device based on the given shopping needs, meal plans, and fitness goals of the user. The system is further individualized to the user because a route may be updated based on an action of the user to deviate from the shopping list and/or meal plan of the user.

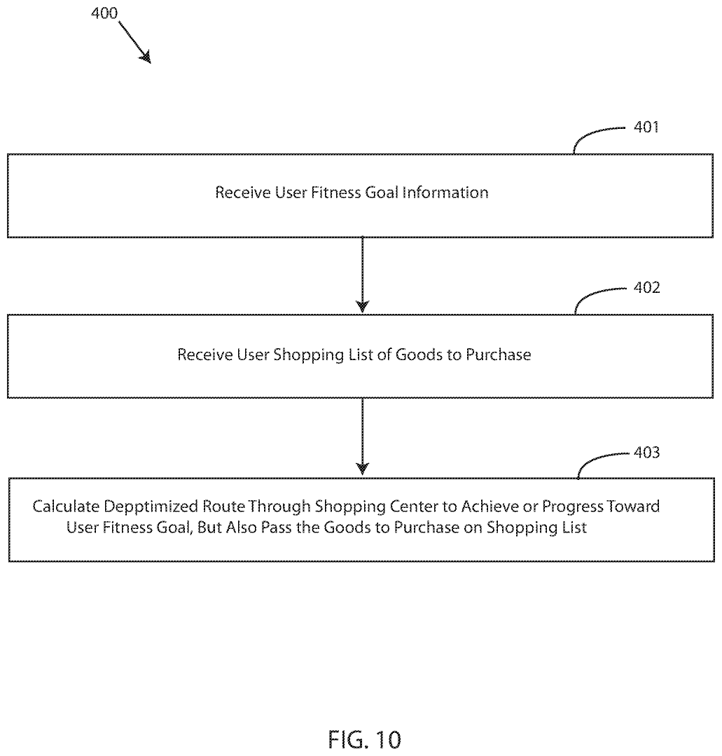

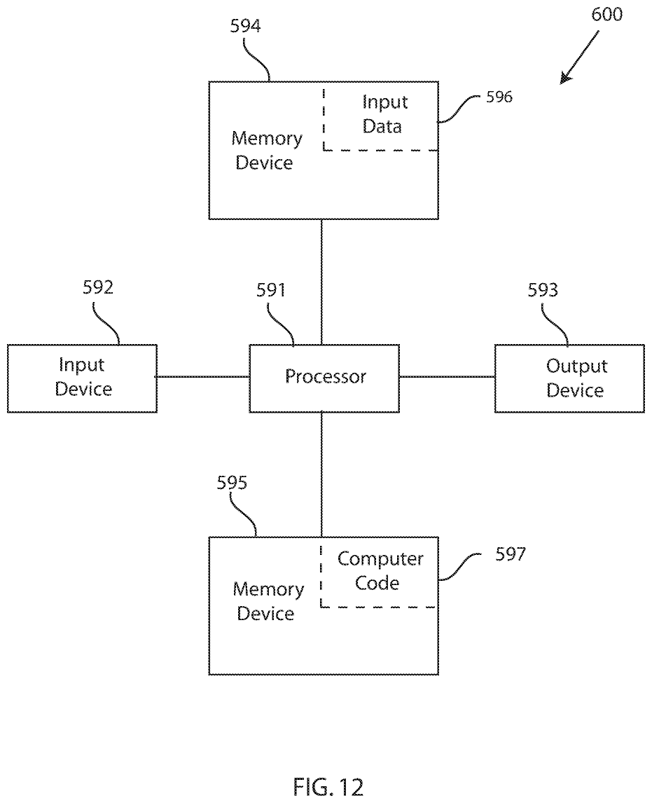

Referring now to FIG. 10, which depicts a flow chart of a first embodiment of a method 400 for generating a health and fitness integrated shopping route throughout a shopping center, in accordance with embodiments of the present invention. One embodiment of a method 400 or algorithm that may be implemented for generating a health and fitness integrated shopping route throughout a shopping center with the route determination system 100 described in FIGS. 1-9 using one or more computer systems as defined generically in FIG. 12 below, and more specifically by the specific embodiments of FIG. 1.

Embodiments of the method 400 for generating a health and fitness integrated shopping route throughout a shopping center, in accordance with embodiments of the present invention, may begin at step 401 wherein a user fitness goal information is received. Step 402 receives the user shopping list of goods to purchase. Step 403 calculates a deoptimized route through a shopping center to achieve, exceed, or progress toward a user fitness goal, but also pass the goods to be purchased on the user's shopping list.

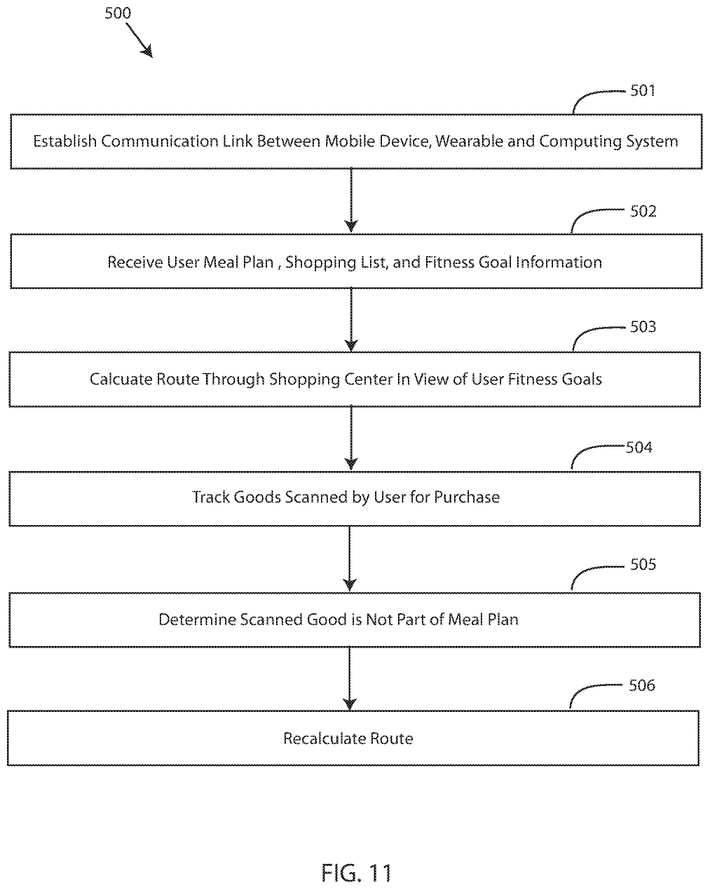

FIG. 11 depicts a flow chart of a second embodiment of a method 500 for generating a health and fitness integrated shopping route throughout a shopping center, in accordance with embodiments of the present invention. Step 501 establishes a communication link between the user mobile device 110, wearable device 111, and the computing system 120. Step 502 receives a user meal plan, shopping list, and fitness goal information. Step 503 calculates a route through the shopping center in view of the user fitness goals. Step 504 tracks goods scanned by the user for purchase. Step 505 determines that a scanned good is not part of the user meal plan. Step 506 recalculates and adjusts the route based on a deviance from the user meal plan.

FIG. 12 depicts a block diagram of a computer system for the route determination system 100 of FIGS. 1-9, capable of implementing methods for generating a health and fitness integrated shopping route throughout a shopping center of FIGS. 10-11, in accordance with embodiments of the present invention. The computer system 600 may generally comprise a processor 591, an input device 592 coupled to the processor 591, an output device 593 coupled to the processor 591, and memory devices 594 and 595 each coupled to the processor 591. The input device 592, output device 593 and memory devices 594, 595 may each be coupled to the processor 591 via a bus. Processor 591 may perform computations and control the functions of computer system 500, including executing instructions included in the computer code 597 for the tools and programs capable of implementing a method for generating a health and fitness integrated shopping route throughout a shopping center in the manner prescribed by the embodiments of FIGS. 10-11 using the route determination system 100 of FIGS. 1-9, wherein the instructions of the computer code 597 may be executed by processor 591 via memory device 595. The computer code 597 may include software or program instructions that may implement one or more algorithms for implementing the method f for generating a health and fitness integrated shopping route throughout a shopping center, as described in detail above. The processor 591 executes the computer code 597. Processor 591 may include a single processing unit, or may be distributed across one or more processing units in one or more locations (e.g., on a client and server).

The memory device 594 may include input data 596. The input data 596 includes any inputs required by the computer code 597. The output device 593 displays output from the computer code 597. Either or both memory devices 594 and 595 may be used as a computer usable storage medium (or program storage device) having a computer-readable program embodied therein and/or having other data stored therein, wherein the computer-readable program comprises the computer code 597. Generally, a computer program product (or, alternatively, an article of manufacture) of the computer system 500 may comprise said computer usable storage medium (or said program storage device).

Memory devices 594, 595 include any known computer-readable storage medium, including those described in detail below. In one embodiment, cache memory elements of memory devices 594, 595 may provide temporary storage of at least some program code (e.g., computer code 597) in order to reduce the number of times code must be retrieved from bulk storage while instructions of the computer code 597 are executed. Moreover, similar to processor 591, memory devices 594, 595 may reside at a single physical location, including one or more types of data storage, or be distributed across a plurality of physical systems in various forms. Further, memory devices 594, 595 can include data distributed across, for example, a local area network (LAN) or a wide area network (WAN). Further, memory devices 594, 595 may include an operating system (not shown) and may include other systems not shown in FIG. 12.

In some embodiments, the computer system 500 may further be coupled to an Input/output (I/O) interface and a computer data storage unit. An I/O interface may include any system for exchanging information to or from an input device 592 or output device 593. The input device 592 may be, inter alia, a keyboard, a mouse, etc. or in some embodiments the touchscreen of a computing device. The output device 593 may be, inter alia, a printer, a plotter, a display device (such as a computer screen), a magnetic tape, a removable hard disk, a floppy disk, etc. The memory devices 594 and 595 may be, inter alia, a hard disk, a floppy disk, a magnetic tape, an optical storage such as a compact disc (CD) or a digital video disc (DVD), a dynamic random access memory (DRAM), a read-only memory (ROM), etc. The bus may provide a communication link between each of the components in computer 500, and may include any type of transmission link, including electrical, optical, wireless, etc.

An I/O interface may allow computer system 500 to store information (e.g., data or program instructions such as program code 597) on and retrieve the information from computer data storage unit (not shown). Computer data storage unit includes a known computer-readable storage medium, which is described below. In one embodiment, computer data storage unit may be a non-volatile data storage device, such as a magnetic disk drive (i.e., hard disk drive) or an optical disc drive (e.g., a CD-ROM drive which receives a CD-ROM disk). In other embodiments, the data storage unit may include a knowledge base or data repository 125 as shown in FIG. 1.

As will be appreciated by one skilled in the art, in a first embodiment, the present invention may be a method; in a second embodiment, the present invention may be a system; and in a third embodiment, the present invention may be a computer program product. Any of the components of the embodiments of the present invention can be deployed, managed, serviced, etc. by a service provider that offers to deploy or integrate computing infrastructure with respect to route determination systems and methods. Thus, an embodiment of the present invention discloses a process for supporting computer infrastructure, where the process includes providing at least one support service for at least one of integrating, hosting, maintaining and deploying computer-readable code (e.g., program code 597) in a computer system (e.g., computer system 500) including one or more processor(s) 591, wherein the processor(s) carry out instructions contained in the computer code 597 causing the computer system for generating a health and fitness integrated shopping route throughout a shopping center. Another embodiment discloses a process for supporting computer infrastructure, where the process includes integrating computer-readable program code into a computer system 500 including a processor.

The step of integrating includes storing the program code in a computer-readable storage device of the computer system 500 through use of the processor. The program code, upon being executed by the processor, implements a method for generating a health and fitness integrated shopping route throughout a shopping center. Thus, the present invention discloses a process for supporting, deploying and/or integrating computer infrastructure, integrating, hosting, maintaining, and deploying computer-readable code into the computer system 500, wherein the code in combination with the computer system 500 is capable of performing a method for generating a health and fitness integrated shopping route throughout a shopping center.

A computer program product of the present invention comprises one or more computer-readable hardware storage devices having computer-readable program code stored therein, said program code containing instructions executable by one or more processors of a computer system to implement the methods of the present invention.

A computer system of the present invention comprises one or more processors, one or more memories, and one or more computer-readable hardware storage devices, said one or more hardware storage devices containing program code executable by the one or more processors via the one or more memories to implement the methods of the present invention.

The present invention may be a system, a method, and/or a computer program product at any possible technical detail level of integration. The computer program product may include a computer-readable storage medium (or media) having computer-readable program instructions thereon for causing a processor to carry out aspects of the present invention.

The computer-readable storage medium can be a tangible device that can retain and store instructions for use by an instruction execution device. The computer-readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer-readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer-readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer-readable program instructions described herein can be downloaded to respective computing/processing devices from a computer-readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer-readable program instructions from the network and forwards the computer-readable program instructions for storage in a computer-readable storage medium within the respective computing/processing device.

Computer-readable program instructions for carrying out operations of the present invention may be assembler instructions, instruction-set-architecture (ISA) instructions, machine instructions, machine-dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer-readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer-readable program instructions.

These computer-readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein comprises an article of manufacture including instructions which implement aspects of the function/act specified in the flowchart and/or block diagram block or blocks.

The computer-readable program instructions may also be loaded onto a computer, other programmable data processing apparatus, or other device to cause a series of operational steps to be performed on the computer, other programmable apparatus or other device to produce a computer-implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein

1. A method for generating a health and fitness integrated shopping route throughout a shopping center, the method comprising:
establishing, by a processor of a computing system, a communication link between at least one of the user mobile device and a user wearable computing device, and the computing system, over a network, wherein the user wearable computing device has at least one sensor for tracking a movement of the user;
in response to establishing the communication link:
receiving, by the processor, from at least one of the user mobile device and the wearable computing device: (i) a meal plan tailored to the user, (ii) a shopping list of the user synchronized with the meal plan to determine a list of goods that the user needs to purchase within the shopping center in accordance with the meal plan, and (iii) a fitness goal of the user that the user has yet to achieve;
calculating, by the processor, a route throughout the shopping center that achieves or exceeds the fitness goal of the user while also guiding the user past the goods on the shopping list of goods that the user needs to purchase, wherein the route prioritizes achieving or exceeding the fitness goal of the user over an optimal route through the shopping center past the list of goods the user needs to purchase;
tracking, by the processor, goods scanned using a mobile payment application for purchase by the user;
determining, by the processor, that a scanned good is not on the shopping list or an approved good of the meal plan, and calculating a net caloric content between the scanned good not on the shopping list and an approved meal plan good being replaced by the scanned good; and
recalculating, by the processor, the route throughout the shopping center to account for the net caloric content.

2. The method of claim 1, further comprising: applying, by the processor, a restriction to at least one wheel of a shopping cart to increase resistance of the shopping cart as the user pushes the shopping cart, to account for some of the net caloric content, wherein the restriction is adjusted based on an acceleration of the shopping cart, a remaining calories to be burned by the user to achieve the fitness goal, and a maximum amount of time the user wishes to spend shopping at the shopping center.

3. The method of claim 1, further comprising: recommending, by the processor, alternatives to the scanned good and the approved meal plan good that result in a lowest net gain caloric content.

4. The method of claim 3, further comprising: displaying, by the processors, a recommendation based on the recommending step, along with a predicted time to complete the updated route.

5. The method of claim 1, further comprising: providing, by the processor, one or more rewards for exceeding the fitness goal of the user.

6. The method of claim 1, wherein the net caloric contents is calculated by comparing, by the processor, a caloric content of the scanned good with a caloric content of a meal plan good being replaced by the scanned good.

7. The method of claim 1, wherein the recalculating includes adding a distance to the route through the shopping center when the net caloric content is a net gain in caloric content.

8. The method of claim 1, wherein the recalculating includes decreasing a distance to the route through the shopping center when the net caloric content is a net loss in caloric content.

9. A computer system, comprising:
a processor;
a memory device coupled to the processor; and
a computer readable storage device coupled to the processor, wherein the storage device contains program code executable by the processor via the memory device to implement a method for generating a health and fitness integrated shopping route throughout a shopping center, the method comprising:
establishing, by a processor of a computing system, a communication link between at least one of the user mobile device and a user wearable computing device, and the computing system, over a network, wherein the user wearable computing device has at least one sensor for tracking a movement of the user;
in response to establishing the communication link:
receiving, by the processor, from at least one of the user mobile device and the wearable computing device: (i) a meal plan tailored to the user, (ii) a shopping list of the user synchronized with the meal plan to determine a list of goods that the user needs to purchase within the shopping center in accordance with the meal plan, and (iii) a fitness goal of the user that the user has yet to achieve;
calculating, by the processor, a route throughout the shopping center that achieves or exceeds the fitness goal of the user while also guiding the user past the goods on the shopping list of goods that the user needs to purchase, wherein the route prioritizes achieving or exceeding the fitness goal of the user over an optimal route through the shopping center past the list of goods the user needs to purchase;
tracking, by the processor, goods scanned using a mobile payment application for purchase by the user;
determining, by the processor, that a scanned good is not on the shopping list or an approved good of the meal plan, and calculating a net caloric content between the scanned good not on the shopping list and an approved meal plan good being replaced by the scanned good; and
recalculating, by the processor, the route throughout the shopping center to account for the net caloric content.

10. The computer system of claim 9, further comprising: applying, by the processor, a restriction to at least one wheel of a shopping cart to increase resistance of the shopping cart as the user pushes the shopping cart, to account for some of the net caloric content, wherein the restriction is adjusted based on an acceleration of the shopping cart, a remaining calories to be burned by the user to achieve the fitness goal, and a maximum amount of time the user wishes to spend shopping at the shopping center.

11. The computer system of claim 9, further comprising: recommending, by the processor, alternatives to the scanned good and the approved meal plan good that result in a lowest net gain caloric content.

12. The computer system of claim 11, further comprising: displaying, by the processors, a recommendation based on the recommending step, along with a predicted time to complete the updated route.

13. The computer system of claim 9, wherein the recalculating includes adding a distance to the route through the shopping center when the net caloric content is a net gain in caloric content.

14. The computer system of claim 9, wherein the recalculating includes decreasing a distance to the route through the shopping center when the net caloric content is a net loss in caloric content.

15. A computer program product, comprising a computer readable hardware storage device storing a computer readable program code, the computer readable program code comprising an algorithm that when executed by a computer processor of a computing system implements a method for generating a health and fitness integrated shopping route throughout a shopping center, comprising:
establishing, by a processor of a computing system, a communication link between at least one of the user mobile device and a user wearable computing device, and the computing system, over a network, wherein the user wearable computing device has at least one sensor for tracking a movement of the user;
in response to establishing the communication link:
receiving, by the processor, from at least one of the user mobile device and the wearable computing device: (i) a meal plan tailored to the user, (ii) a shopping list of the user synchronized with the meal plan to determine a list of goods that the user needs to purchase within the shopping center in accordance with the meal plan, and (iii) a fitness goal of the user that the user has yet to achieve;
calculating, by the processor, a route throughout the shopping center that achieves or exceeds the fitness goal of the user while also guiding the user past the goods on the shopping list of goods that the user needs to purchase, wherein the route prioritizes achieving or exceeding the fitness goal of the user over an optimal route through the shopping center past the list of goods the user needs to purchase;
tracking, by the processor, goods scanned using a mobile payment application for purchase by the user;
determining, by the processor, that a scanned good is not on the shopping list or an approved good of the meal plan, and calculating a net caloric content between the scanned good not on the shopping list and an approved meal plan good being replaced by the scanned good; and
recalculating, by the processor, the route throughout the shopping center to account for the net caloric content.

16. The computer program product of claim 15, further comprising: applying, by the processor, a restriction to at least one wheel of a shopping cart to increase resistance of the shopping cart as the user pushes the shopping cart, to account for some of the net caloric content, wherein the restriction is adjusted based on an acceleration of the shopping cart, a remaining calories to be burned by the user to achieve the fitness goal, and a maximum amount of time the user wishes to spend shopping at the shopping center.

17. The computer program product of claim 15, further comprising: recommending, by the processor, alternatives to the scanned good and the approved meal plan good that result in a lowest net gain caloric content.

18. The computer program product of claim 17, further comprising: displaying, by the processors, a recommendation based on the recommending step, along with a predicted time to complete the updated route.

19. The computer program product of claim 15, wherein the recalculating includes adding a distance to the route through the shopping center when the net caloric content is a net gain in caloric content.

20. The computer program product of claim 15, wherein the recalculating includes decreasing a distance to the route through the shopping center when the net caloric content is a net loss in caloric content.
